(19) United States
(12) Patent Application Publication
(10) Pub. No.: US 2014/0200366 A1
(43) Pub. Date: Jul. 17, 2014

(54) METHOD FOR REDUCING FOULING DURING PURIFICATION OF (METH)ACRYLATE ESTERS

(71) Applicants: Michael A. Curtis, Houston, TX (US); Michael S. Decourcy, Houston, TX (US); David A. Flosser, Missouri City, TX (US); Melissa Harris, Sugar Land, TX (US); Jamie J. Juliette, Houston, TX (US); Philippe P. Maillot, Kingwood, TX (US)

(72) Inventors: Michael A. Curtis, Houston, TX (US); Michael S. Decourcy, Houston, TX (US); David A. Flosser, Missouri City, TX (US); Melissa Harris, Sugar Land, TX (US); Jamie J. Juliette, Houston, TX (US); Philippe P. Maillot, Kingwood, TX (US)

(73) Assignee: ROHM AND HAAS COMPANY, Philadelphia, PA (US)

(21) Appl. No.: 14/119,904

(22) PCT Filed: Jun. 1, 2012
(86) PCT No.: PCT/US2012/040349

§ 371 Date: Jan. 16, 2014

Related U.S. Application Data
(60) Provisional application No. 61/492,960, filed on Jun. 3, 2011.

(51) Int. Cl. C07C 51/44

(52) U.S. Cl. CPC C07C 51/44
USPC 562/600

(57) ABSTRACT

The present invention provides a method for reducing accumulation of solid materials when manufacturing a (meth)acrylic acid ester having low biacetyl content (less than 2 ppm) by adding an aromatic diamine under conditions which provide sufficient residence time and thorough mixing to react up to 100% by weight of the biacetyl in the crude (meth)acrylic acid ester stream, prior to separation and purification. A feedback method is also provided for reducing solids accumulation in the separation and purification equipment of such processes by measuring the biacetyl content and adjusting the aromatic diamine addition rate so that excess aromatic diamine can be minimized. A third embodiment provides a method for reversing an accumulation of solid materials during such processes, while still producing a (meth)acrylic acid ester having low biacetyl content (less than 2 ppm), by reducing or ceasing the addition rate of aromatic diamine for a period of time.

### FIELD OF THE INVENTION

The present invention relates to a method for reducing fouling of downstream apparatus during purification of (meth)acrylate esters, particularly where aromatic amines are present.

### BACKGROUND OF THE INVENTION

(Meth)acrylic acid esters, such as methyl acrylate, methyl methacrylate, ethyl acrylate, ethyl methacrylate, butyl acrylate, and butyl methacrylate, are useful for production of specialty polymer compositions such as, for example, superabsorbent polymers, acrylic binders, as well as for polymers efficient as dispersants for oil well drilling muds, flocculating agents and making flat panel displays. Impurities are typically present in (meth)acrylic acid esters that may interfere with polymerization reactions, or adversely impact polymer properties including hardness, color and elasticity. Thus, processes and methods for purifying (meth)acrylic acid esters, i.e., separating the desired (meth)acrylate ester product from other product stream components, are of critical importance in the production of specialty polymer grade (i.e., at least 99% pure) (meth)acrylate esters.

There are various commercially-practiced processes for producing (meth)acrylic acid esters, all of which produce a mixed product stream which is often referred to as “crude” (meth)acrylic acid ester. A crude (meth)acrylic acid ester stream typically contains not only the desired (meth)acrylic acid ester, but also water and various other impurities including, without limitation, unreacted compounds, impurities introduced with raw materials, as well as intermediate and side products. Depending on which (meth)acrylic acid ester is manufactured and which process is practiced, such impurities may include, without limitation, one or more alcohols such as methanol, one or more aldehyde compounds such as acrolein, maleic anhydride, and furfural, as well as one or more carbonyl compounds such as biacetyl.

Crude (meth)acrylic acid ester streams are generally subjected to one or more separation and purification processes to remove water and other impurities such as those mentioned above. After one or more separation steps are performed to remove a portion of the water and, optionally, at least some of the unreacted raw materials so they can be recycled to the process or used in other processes, the resulting “stripped” crude (meth)acrylic acid ester may be subjected to one or more additional separation and purification steps, such as distillation, wherein the desired (meth)acrylate acid ester is separated from heavier and higher-boiling compounds to produce an overhead distilled (meth)acrylate acid ester product stream and a bottoms stream comprising the heavier, high boiling compounds and a small amount of the (meth)acrylate acid ester. The bottoms stream may be subjected to further purification in another separation step to recover a portion of the (meth)acrylate acid ester still present in this stream to produce an overhead distilled (meth)acrylate acid ester stream and a further concentrated bottoms stream containing heavier compounds, which may be discarded as waste or burned as fuel.

Although a stripped crude (meth)acrylate acid ester stream generally contains remaining impurities in relatively small amounts (e.g., less than a few weight percent, or even in the parts per million range), certain impurities are known particularly, even in small amounts, to interfere with the properties of specialty polymers subsequently manufactured from (meth)acrylic acid ester monomers. For example, biacetyl (2,3-butanedione) present in methyl methacrylate, in an amount of greater than about 5 ppm (parts per million, by weight), is known to cause discoloration in the final polymer products. Various additives known to facilitate removal of one or more of such detrimental impurities are, therefore, sometimes added to the manufacturing process at one or more points, such as during reaction steps or separation and purification steps.

The manufacture of methyl methacrylate (MMA), for example, may be accomplished by a variety of processes, one of which is a multi-step reaction process beginning with reaction of acetone cyanohydrin (ACH) and sulfuric acid and ending with esterification (hereinafter referred to as the “conventional ACH route to MMA”) to form a crude MMA stream. Another process involves sequential oxidation of isobutylene (or tert-butyl alcohol) to methacrolein, and then to methacrylic acid, which is then esterified with methanol to produce crude methyl methacrylate (hereinafter referred to as the “conventional C₄-based process” for producing MMA). Additionally, a crude methyl methacrylate stream may be produced by carbonylation of propylene in the presence of acids to produce isobutyric acid, which is then dehydrogenated (hereinafter referred to as the “conventional C₃-based process” for producing MMA). Of course, there are other various processes known and practiced for manufacturing other kinds of (meth)acrylic acid esters.

It is known that addition of one or more amine compounds to a process for manufacturing MMA facilitates the removal and separation of aldehyde and carbonyl impurities from the MMA product. See, U.S. Pat. Nos. 5,571,386 and 6,228,227. Suitable amine compounds include, for example, without limitation, monoethanolamine (“MEA”), ethylenediamine, diethylenetriamine, dipropylenetriamine, and ortho-, para-, and meta-phenylenediamine (i.e., “oPD”, “pPD”, and “mPD”). Generally, it is believed that such amine compounds react and combine with one or more impurity compounds to form adducts which are heavier and have higher boiling points than the originally present impurities, as well as the MMA, which facilitates separation in one or more conventional distillation steps.

As described in U.S. Pat. No. 4,668,818, it is also known to provide a hydrazine or an aromatic ortho-diamine to the esterification reaction mixture of a conventional ACH route to MMA process, to facilitate separation and removal of biacetyl during the subsequent downstream purification steps. It is explained in U.S. Pat. No. 4,668,818 that the aromatic ortho-diamine should be added at a molar ratio of aromatic ortho-diamine to biacetyl of from 1:1 to 200:1, preferably 20:1, in the presence of a strong acid catalyst such as sulfuric acid, such as during or immediately after esterification.

DeCourcy, et al., “Purification of Methacrylic Acid Esters,” Research Disclosure Database Number 544006, August 2009, describes a method for removing biacetyl from stripped crude MMA using one or more aromatic amines (e.g., mPD, oPD, and pPD) in a molar ratio of aromatic diamine to biacetyl of not more than about 10:1, which is significantly less than previously added to the esterification step, and accomplishes a comparable degree of biacetyl removal as described in U.S. Pat. No. 4,668,818. DeCourcy, et al. explain that the aromatic amine should be added subsequent to the esterification step, such as, for example, to the stripped crude product stream (i.e, after the esterification step and before purification of the stripped crude stream). Furthermore, the aromatic amine may be added to process streams in between any two of the separation steps, or even to the equipment in which one or more of the separation steps is being performed.

Unfortunately, addition of aromatic amines in excess of the amount necessary to react with the biacetyl present in the MMA not only results in unnecessary raw material expenses, but also results in fouling (accumulation of solid materials) of the equipment used in the separation and purification steps, which in turn decreases the efficiency of the MMA production process. The equipment observed to be subject to such fouling includes, without limitation, stripping columns, distillation columns, reboilers, condensers, and heat exchangers, as well as the pipes and other lines connecting such equipment. For example, U.S. Pat. No. 5,585,514, explains specifically that aromatic ortho-diamines cause fouling of downstream distillation column heating pipes and, therefore, the use of non-aromatic 1,2-diamines is preferred for biacetyl removal from crude MMA.

Thus, it would be advantageous to have some way of reducing the fouling that occurs in downstream purification equipment of MMA manufacturing processes where an aromatic diamine is being added to facilitate removal of biacetyl, while still maintaining a degree of purification that produces the required purity grade of MMA product. The present invention addresses this need.

### SUMMARY OF THE INVENTION

The methods of the present invention reduce accumulation of solid material in separation and purification equipment in a process for producing a (meth)acrylic acid ester having a biacetyl content of less than 2 parts per million (ppm), where the process comprises providing a crude (meth)acrylic acid ester stream comprising: at least 95% (meth)acrylic acid ester, not more than 5% water, and not more than 50 ppm biacetyl, by weight, based on the total weight of the crude (meth)acrylic acid ester stream and adding an aromatic diamine to the crude (meth)acrylic acid ester stream at an addition rate which produces a treated crude (meth)acrylic acid ester stream, and reacting at least a portion of the total biacetyl present in the crude (meth)acrylic acid ester stream with the aromatic diamine. After reacting at least a portion of the biacetyl with the aromatic diamine, the treated crude (meth)acrylic acid ester stream is distilled in the separation and purification equipment to produce an overhead product which is a purified (meth)acrylic acid ester stream comprising at least 99% by weight (meth)acrylic acid ester, not more than 1% by weight water, and less than 2 ppm biacetyl, based on the total weight of the purified (meth)acrylic acid ester stream. The aromatic diamine comprises at least one compound selected from the group consisting of: ortho-phenylenediamine, para-phenylenediamine, and meta-phenylenediamine. The (meth)acrylic acid ester may be methyl methacrylate. In (meth)acrylic acid ester production processes where the aromatic diamine is added at an addition rate which produces a treated crude (meth)acrylic acid ester stream having an initial molar ratio of aromatic diamine to biacetyl of not more than 10:1, the method of the present invention comprises performing the step of reacting at least a portion of the biacetyl with the aromatic diamine prior to distilling the treated crude (meth)acrylic acid stream by (1) adding the aromatic amine far enough upstream of the separation and purification equipment to provide a residence time of between 10 and 1200 seconds for the aromatic amine to contact biacetyl in the crude (meth)acrylic acid ester stream before performing the distilling step, and (2) thoroughly mixing the aromatic diamine with the crude (meth)acrylic acid ester stream. Furthermore, in accordance with the method of the present invention, thoroughly mixing (2) the aromatic diamine with the crude (meth)acrylic acid ester stream is accomplished by at least one of the following techniques:
a) operating the process with a flow rate of crude (meth)acrylic acid ester stream sufficient to provide turbulent flow conditions, which comprises having a Reynolds number greater than 4000, in the process equipment, and
b) providing the crude (meth)acrylic acid stream and the aromatic amine, or the treated crude (meth)acrylic acid ester stream, to apparatus positioned upstream of the separation and purification equipment and having mixing means comprising one or more static mixers, baffles, recirculation loops, agitators, powered in-line mixers, and mechanical mixers.

The apparatus positioned upstream of the separation and purification equipment comprises a vessel, a pipe, a conduit, a tank, or a combination thereof.

In (meth)acrylic acid ester production processes where the aromatic diamine is added at an addition rate which produces a treated crude (meth)acrylic acid ester stream having an initial molar ratio of aromatic diamine to biacetyl between 1:1 and 100:1, another method in accordance with the present invention comprises adjusting the addition rate of the aromatic diamine during operation of the separation and purification equipment by (i) monitoring the biacetyl content of the purified (meth)acrylic acid ester stream to obtain a measured value biacetyl content; and (ii) taking one of the following actions depending upon how the measured value biacetyl content compares to the target biacetyl content:
(a) maintaining the addition rate at its current value while the measured biacetyl concentration is between a predetermined lower limit and a predetermined upper limit;
(b) increasing the addition rate when the measured value biacetyl content is greater than the upper limit; and
(c) decreasing the addition rate when the measured value biacetyl content is less than the lower limit.

When the addition rate of the aromatic diamine is adjusted by decreasing the addition rate, the addition rate may be maintained at zero for a period of time and then increased above zero.

In (meth)acrylic acid ester production processes where the aromatic diamine is added at an addition rate which produces a treated crude (meth)acrylic acid ester stream having an initial molar ratio of aromatic diamine to biacetyl between 1:1 and 100:1, another embodiment of the method of the present invention is for reversing accumulation of solid material in separation and purification equipment of such processes, and the method comprises determining that solid material has accumulated to an unacceptable degree in the separation and purification equipment by monitoring at least one operating condition and observing said at least one operating condition falling outside a predetermined acceptable range; and reducing and maintaining the addition rate of aromatic diamine within a range of values less than a set addition rate, for a period of time until said at least one operating condition is observed to fall within said predetermined acceptable range. The range of values less than the set addition rate may have a lower limit of zero. The overhead product, which is a purified (meth)acrylic acid ester stream, may be accumulated and blended in one or more tanks to homogenize the biacetyl concentration therein.

### BRIEF DESCRIPTION OF THE DRAWINGS

A more complete understanding of the present invention will be gained from the embodiments discussed hereinafter and with reference to the accompanying drawing, wherein:

FIG. 1 is a schematic representation of a process for further purification of stripped crude (meth)acrylate to which the present invention is applicable; and

FIG. 2 is a schematic representation of the commercial-scale MMA distillation system used to perform the commercial scale examples provided herein.

### DETAILED DESCRIPTION OF THE INVENTION

Initially, it is noted that in the following description, endpoints of ranges are considered to be definite and are recognized to incorporate within their tolerance other values within the knowledge of persons of ordinary skill in the art, including, but not limited to, those which are insignificantly different from the respective endpoint as related to this invention (in other words, endpoints are to be construed to incorporate values “about” or “close” or “near” to each respective endpoint). The range and ratio limits, recited herein, are combinable. For example, if ranges of 1-20 and 5-15 are recited for a particular parameter, it is understood that ranges of 1-5, 1-15, 5-20, or 15-20 are also contemplated and encompassed thereby.

The present invention provides methods for reducing, and even reversing, the accumulation of solid materials (i.e., “fouling”) in separation and purification equipment. This problem is often caused by the use of aromatic diamines in processes for producing (meth)acrylic acid esters. For example, as discussed previously, aromatic diamines are sometimes used to facilitate separation and removal of the carbonyl compound biacetyl from crude (meth)acrylic acid esters. Thus, regardless of the particular manufacturing process practiced, the present invention may be beneficially applied to purification processes that produce high purity (meth)acrylic acid esters from crude (meth)acrylic acid esters which comprise biacetyl, wherein an aromatic diamine is added during either manufacture or further separation and purification of a crude (meth)acrylic acid ester.

In particular, a first embodiment of the present invention is a method relating to reducing accumulation of solid materials in the separation and purification equipment of such processes while still producing a (meth)acrylic acid ester having low biacetyl content (e.g., from 0 ppm to less than 2 ppm) by adding an aromatic diamine under conditions which provide sufficient residence time and thorough mixing to reduce the biacetyl content to a value less than 2 ppm, prior to performing separation and purification. Another embodiment of the present invention provides a method for adjusting the aromatic diamine addition rate depending upon measuring the biacetyl content of the distilled (meth)acrylic acid ester product so that the excess aromatic diamine can be minimized even when the biacetyl content of the crude (meth)acrylic acid ester fluctuates.

A third embodiment of the present invention is a method relating to reversing an accumulation of solid materials in the separation and purification equipment of such processes, while still producing a (meth)acrylic acid ester having low biacetyl content (e.g., from 0 ppm to less than 2 ppm), by reducing or ceasing the addition rate of aromatic diamine for a period of time when an unacceptable degree of solid material accumulation is detected by monitoring relevant operating conditions.

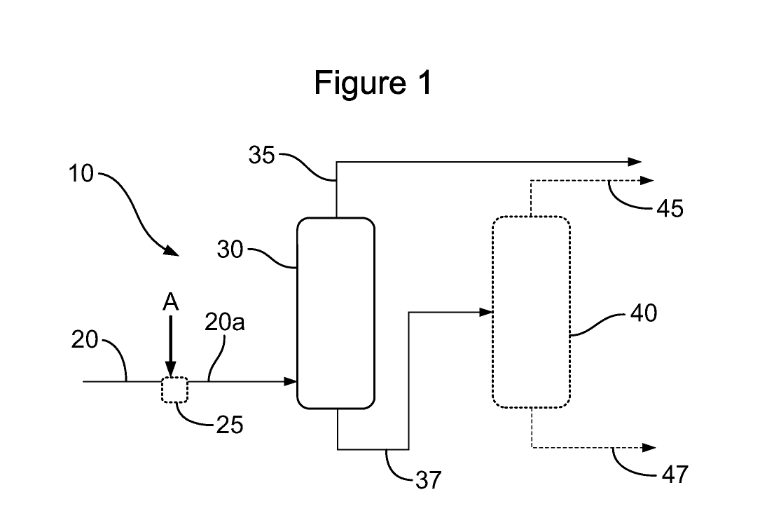

With reference now to FIG. 1, a schematic diagram is provided showing the steps involved in a general process 10 for purifying a crude (meth)acrylic acid ester stream 20. In order to focus more clearly on the separation and purification steps (30,40) which are most relevant to the present invention, upstream processes and steps, such as reactions and optional preliminary water removal steps, for manufacturing the crude (meth)acrylic acid ester stream are omitted from FIG. 1. Regardless of the particular manufacturing process employed to produce it, after production and, optionally, an initial separation step such as stripping low boiling point raw materials, further purification of the crude (meth)acrylic acid ester stream 20 is typically performed in a purification process 10 having one or more separation and purification steps 30, 40. As already understood by persons of ordinary skill in the relevant art, the separation and purification steps 30, 40 are performed using separation and purification equipment (not shown per se) including, without limitation, one or more distillation columns, strippers, mixing vessels, reservoirs, rectification columns, gravity separators, condensers, reboilers coolers, and other equipment suitable for treating process streams to separate the desired (meth)acrylic acid ester from other components of the crude stream 20.

While the various embodiments of the present invention will, hereinafter, be described in detail as applied to a process for the production of high purity methyl methacrylate (MMA) (i.e., having at least 99% by weight MMA and from 0 to less than 2 ppm biacetyl), it should be understood that the present invention is applicable to processes for producing other types of (meth)acrylic acid esters, including without limitation, methyl acrylate, ethyl acrylate, ethyl methacrylate, butyl acrylate, and butyl methacrylate. Furthermore, the present invention is suitable for use with crude (meth)acrylic acid streams derived from any manufacturing process. For example, although the crude MMA stream described hereinafter was produced by a process following the conventional ACH route to MMA, the crude MMA stream could have been derived from the conventional C₃- or C₄-based processes.

With reference now back to FIG. 1, typically, a stripped crude methyl methacrylate (MMA) stream 20 will be fed to the separation and purification process 10 for further purification, including but not limited to, separation and removal of biacetyl. This stripped crude MMA stream 20 has already been subjected to a stripping step and, therefore, should comprise at least 95% MMA, not more than 5% water, and not more than 50 ppm biacetyl, by weight, based on the total weight of the crude MMA stream 20. For example, without limitation, the stripped crude MMA stream 20 may comprise not more than 25 ppm biacetyl, or even not more than 10 ppm biacetyl. The stripped crude MMA stream 20 may further comprise one or more other impurities such as, without limitation, water, methacrylic acid, methanol, acrolein, maleic anhydride, furfural, and formaldehyde.

More particularly, the stripped crude MMA stream 20 may be subjected to a first distillation step 30 wherein at least a portion of the desired MMA product is separated from heavier and higher-boiling compounds to produce an overhead purified MMA stream 35 (also referred to as a “distilled MMA stream,” or “DMMA stream,” 35), and a heavy ends stream 37 comprising the heavier, high boiling compounds and a small amount of MMA. The purified MMA stream (DMMA stream) 35 comprises at least 99% MMA, not more than 1% water, and between 0 and less than 2 ppm biacetyl, by weight, based on the total weight of the purified MMA stream 35. The heavy ends stream 37 comprises less than 60% by weight of the (meth)acrylic acid ester and compounds having boiling points greater than that of the (meth)acrylic acid ester (such as the reaction product of biacetyl and the aromatic diamine), based on the total weight of the stream 37.

The bottoms stream 37 may be subjected to additional purification in a second distillation step 40 (optional and, therefore, shown in phantom in FIG. 1) to recover a portion of the MMA still present in this stream. Such a second distillation step 40 typically produces a second purified MMA stream 45 (also referred to as a distilled MMA stream, or DMMA stream, 45) which also comprises at least 99% MMA, not more than 1% water, and from 0 to less than 2 ppm biacetyl, by weight, based on the total weight of the second purified MMA stream 45. A further concentrated second heavy ends stream 47, containing heavier compounds, is also produced by the second distillation step 40 and may be discarded as waste or burned as fuel.

As explained hereinabove, in order to remove carbonyl impurities such as biacetyl, one or more aromatic diamines would conventionally have been added to one or more of the steps performed to produce the crude MMA stream, such as during an esterification step (not shown) or after esterification but prior to a stripping step (not shown), or even after stripping (i.e., to the crude MMA stream 20 shown in FIG. 1), in molar ratios of aromatic diamine to biacetyl between 1:1 and 100:1. In practice, a molar ratio of aromatic diamine to biacetyl of about 20:1 has been necessary to achieve the desired biacetyl content of less than 2 ppm in the purified MMA streams 35, 45. However, as also discussed previously, this practice has resulted in the aforementioned fouling of the separation and purification equipment used to perform the further purification 10. This has been particularly true when the aromatic diamine was an aromatic ortho-diamine.

As previously explained, the aromatic diamine facilitates separation of the biacetyl from the MMA by conventional distillation by reacting with the biacetyl to form a compound which is heavier and has a higher boiling point than either the biacetyl or MMA. Thus, as will be understood by persons of ordinary skill in the relevant art, sufficient residence time and thorough mixing in the upstream apparatus, prior to the further purification 10 of the crude MMA stream 20, become critical to ensure that enough biacetyl will be converted (i.e., reacted with aromatic diamine to form the heavier compound), prior to further purification steps 30, 40, to enable its removal and production of a DMMA product (e.g., DMMA stream 35 shown in FIG. 1) having less than 2 ppm biacetyl.

The molar ratio of aromatic diamine to biacetyl of about 20:1 conventionally used in MMA production processes represents an amount of aromatic diamine in large excess of the amount necessary (i.e., a molar ratio of 1:1) to convert substantially all of the biacetyl present in the stripped crude MMA stream 20 to a compound more easily removed during distillation. Without wishing to be bound by theory, it is believed that it is the presence of excess aromatic diamine (i.e., aromatic diamine not consumed by conversion of biacetyl to heavier compounds) in the stripped crude MMA stream that results in fouling of equipment during further purification 10. Surprisingly, it has been determined that an aromatic diamine may be added in a lower molar ratio (i.e., not more than 10:1) than previously believed necessary to produce an MMA product having from 0 to less than 2 ppm biacetyl, as long as the aromatic diamine is added under conditions which allow a sufficient portion of the total biacetyl present in the stripped crude MMA stream 20 to be converted prior to being further purified, such as in the first distillation step 30. For example, where the stripped crude MMA stream 20 comprises 50 ppm biacetyl, the “sufficient portion” to be converted would be 96% of the biacetyl, leaving not more than 2 ppm in the treated crude MMA stream 25a. If the stripped crude MMA stream 20 comprises 10 ppm biacetyl, producing a purified MMA stream having less than 2 ppm biacetyl would require converting 80% of the biacetyl. Thus, the “sufficient portion” of biacetyl to be converted in the stripped crude MMA stream 20 is readily calculable by persons of ordinary skill in the relevant art.

As described in greater detail hereinafter, “sufficient residence time” is from 10 to 1200 seconds and can be ensured by selecting an addition point far enough upstream of the further purification process 10 that the aromatic diamine and biacetyl are in contact with one another for a period between 10 to 1200 seconds. This method is further enhanced by sufficiently mixing the aromatic diamine with the stripped crude MMA stream also prior to the further purification process 10.

Thus, in one embodiment of the present invention, an aromatic diamine is added to the stripped crude MMA stream 20, at a molar ratio of aromatic diamine to biacetyl of not more than 10:1, and at a point far enough upstream of the further purification process 10 to provide from 10 to 1200 seconds of residence time. This produces a treated crude MMA stream 20a having less biacetyl than in the stripped crude MMA stream 20. In other words, the aromatic diamine is added to the stripped crude MMA stream 20, at a point downstream of, or subsequent to, the manufacture of the stripped crude MMA stream 20, but far enough upstream of the further purification process 10 to provide a residence time of 10 to 1200 seconds.

Suitable aromatic diamines include, for example, ortho-phenylenediamine (oPD), para-phenylenediamine (pPD), and meta-phenylenediamine (mPD). The aromatic diamine may be added neat (i.e., at least 99% pure), however, as is readily apparent to persons of ordinary skill, preparing a solution comprising the aromatic diamine and a solvent and then adding the diamine-containing solution to the stripped crude MMA stream 20 will provide faster and more homogenous mixing of the aromatic diamine in the MMA streams. For example, without limitation, the solution may comprise from 0.5% to 8% by weight of aromatic diamine, based on the total weight of the solution, and the solvent would be the same as the particular (meth)acrylic acid ester product (e.g., MMA). Hereinafter, any reference to adding or feeding aromatic diamine includes using neat aromatic diamine or using a solution comprising 0.5% to 8% by weight of aromatic diamine, based on the total weight of the solution, as described above.

More particularly, the aromatic diamine should be added upstream of, or prior to, the first distillation step 30. More particularly, without limitation, the aromatic diamine may be added at a molar ratio of aromatic diamine to biacetyl of not more than 10:1 to the stripped crude MMA stream 20, such as proximate to the position indicated by arrow A in FIG. 1, to produce a treated crude MMA stream 20a, which is then subjected to the first distillation step 30. In addition to adding the aromatic diamine upstream of the first distillation step 30, the aromatic diamine may also be added to the MMA stream at other points during further distillation 10, such as downstream of (i.e., subsequent to) the first distillation step 30 but upstream of (i.e., prior to) the second distillation step 40. More particularly, without limitation, the aromatic diamine may be added at a molar ratio of aromatic diamine to biacetyl of not more than 10:1 to the heavy ends stream 37 which exits the first distillation step 30. The second purified MMA stream 45 produced in this manner would also comprise at least 99% MMA, not more than 1% water, and from 0 less than 2 ppm biacetyl, by weight, based on its total weight.

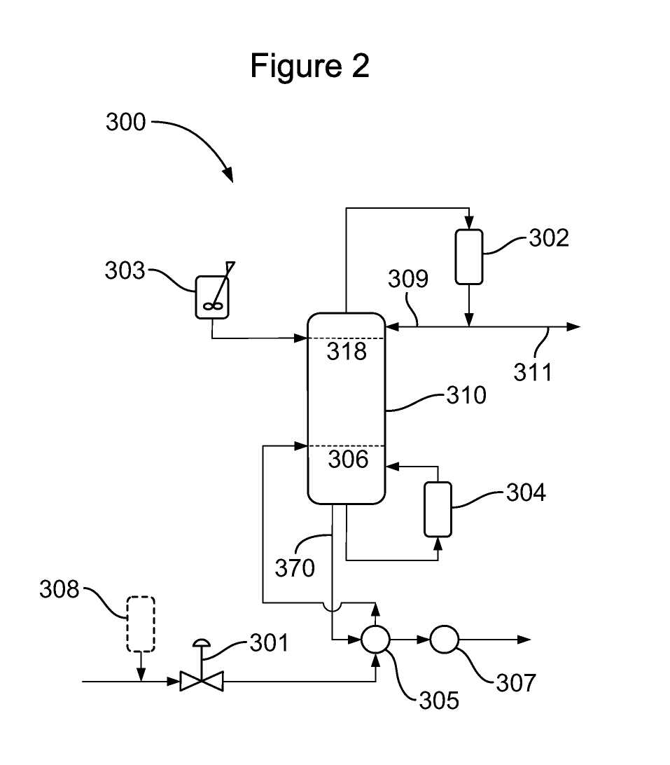

In practice, the aromatic diamine is fed (added) to apparatus positioned upstream of the separation and purification equipment used to perform the further purification 10 of the stripped crude MMA stream 20. The upstream apparatus may be, without limitation, one or more of: a vessel, a pipe, a conduit, and a tank (e.g., see mixing tank 25 shown in phantom in FIG. 1 described in detail below). Furthermore, in accordance with the method of the present invention, the apparatus may have mixing means comprising one or more static mixers, baffles, recirculation loops, agitators, powered in-line mixers, and mechanical mixers (not shown per se in FIG. 1, but see FIG. 2).

The concept of residence time is well known to persons of ordinary skill in the relevant art and is generally understood to be the average amount of time that a particular particle spends in a particular system, or in a particular volume within a system. The bounds of the system or volume within the system may be arbitrarily chosen to fit the particular process or equipment being assessed, but once defined it must remain the same throughout characterization. In other words, residence time depends directly on the amount of substance that is present and begins from the moment that the particle of a particular substance enters the volume and ends the moment that the same particle of that substance leaves the volume. If the volume changes, then the residence time will also change, assuming the rates of flow of the substance into and out of the volume are held constant. For example, the larger the volume, then the greater the residence time and, similarly, the smaller the volume, the shorter the residence time will be. Additionally, as will be recognized by persons of ordinary skill, if the rates of flow in and out of the volume are increased, the residence time will be shorter. If the rates of flow of the substance in and out of the volume are decreased, then the residence time will be longer. This is, of course, assuming that the concentration of the substance in the system (or volume) and the size of the system (or volume) remain constant, and assuming steady-state.

As used herein and with reference to FIG. 1, the residence time means a period of time, prior to being subjected to further purification 10, and during which the aromatic diamine and biacetyl are both in contact with one another, in the same one of one or more of the process streams, such as in the stripped crude MMA stream 20, prior to entering the first distillation step 30.

As will be appreciated by persons of ordinary skill in the relevant art, there are various techniques for achieving thorough mixing of the treated crude MMA stream 20a and selection of which technique is appropriate and effective depends upon the physical nature of the reaction system in use. More particularly, when the treated crude MMA stream 20a is flowing in a pipe or conduit, thorough mixing, as used herein, means that the MMA stream has turbulent flow conditions, which requires a Reynolds number greater than 4,000, during the residence time. As will be familiar to persons of ordinary skill in the relevant art, the Reynolds number is a dimensionless number which is calculated based on the physical parameters of a system and the actual fluid flow therethrough. The value of the Reynolds number calculated for a particular pipe allows us to characterize the flow regime as laminar or turbulent. Laminar flow is characterized by smooth, constant fluid motion, in a system where viscous forces are dominant. Turbulent flow is dominated by inertial forces, which tend to produce chaotic eddies, vortices and other flow instabilities, which promote thorough mixing of fluid components. When the system is a pipe, laminar flow occurs when the Reynolds number is less than 2300, and turbulent flow occurs when the Reynolds number is greater than 4000. In the interval between 2300 and 4000, laminar and turbulent flows are possible (′transition′ flows), depending on other factors, such as pipe roughness and flow uniformity:

The following is an example of the calculation of a Reynolds number for fluid flowing through a pipe, and is not intended to limit the present invention in any way.

$$Reynolds Number = \frac{D v p}{u}$$

where D is the inner diameter of the pipe (in meters or feet), v is velocity of the fluid in the pipe (in meters or feet per second), p is density of the fluid (in kilograms per cubic meter or pounds per cubic foot), and u is the viscosity of the fluid (in kilogram meters per second or pound feet per second). If we have a pipe containing flowing fluid and having the following parameters:

$$D = 0.1023 meter ( 0.3355 feet ) , \\ v = 1.12 meters \text{/} sec ( 3.66 fps ) , \\ p = 935.55 kg \text{/} cubic meter ( 58.4 lb \text{/} {ft}^{3} ) , and$$

$$u = 0.0005 kg \text{-} m \text{/} sec ( 0.000336 lb \text{/} ft \text{-} sec = 0.5 centipoise ) , \\ then R = \frac{( 0.1023 ) ( 1.12 ) ( 935.55 )}{( 0.0005 )} = 214 \text{,} 383$$

Since 214,383 is clearly greater than 4,000, it can be concluded that the flow in the above described pipe is turbulent and, therefore, that thorough mixing of the components of the fluid therein is occurring in accordance with the present invention. When the stripped crude MMA stream 20 and aromatic diamine fed to a tank or other vessel for mixing and reaction time to produce a treated crude MMA stream 20a which flows therefrom, thorough mixing, as used herein, means that the vessel or tank has mechanical internal mixing means to enhance intimate contact between biacetyl contained in the stripped crude MMA stream 20 and the aromatic diamine during the time the treated crude MMA stream 20a is contained in the tank or other vessel.

To provide sufficient residence time, as described above in accordance with the method of the present invention, the aromatic diamine may be added or fed to apparatus (not shown in FIG. 1 per se) positioned upstream of the further purification process 10 and which contains or is fed at least a portion of the stripped crude MMA stream 20. The upstream apparatus may include, for example, one or more of a vessel, a pipe, a conduit, or a tank. Of course, if the upstream apparatus has mixing means (such as an agitator, baffle, or mechanical stirrer), the mixing of the aromatic diamine in the crude MMA stream is enhanced.

With reference again to FIG. 1, for example, the stripped crude MMA stream 20 may be fed to a mixing tank 25 (optional and, therefore, shown in phantom) having one or more internal mechanical agitators (not shown), and the aromatic amine may also be fed, in a molar ratio of aromatic diamine to biacetyl of not more than 10:1, to the mixing tank 25, where they are thoroughly mixed together with a residence time of at least 10 seconds, before being fed to the further purification process 10 (e.g., the first distillation step 30). The molar ratio of aromatic diamine to biacetyl in the mixing tank 25 may be, for example, no more than 2:1, or even no more than 5:1.

The initial biacetyl content of the stripped crude MMA stream 20 should typically be no more than 50 ppm, for example, without limitation, no more than 25 ppm, or even no more than 10 ppm. In such circumstances, the residence time of the aromatic diamine and stripped crude MMA stream 20 in the mixing tank 25 may be between 10 and 1200 seconds, for example, at least 300 seconds, or even at least 600 seconds. Where no tank is present and the same biacetyl content parameters are present, the aromatic diamine may be fed directly to a pipe in which the stripped crude MMA stream 20 is being conveyed, but under turbulent flow (i.e., thorough mixing, as described hereinabove in connection with a Reynolds number greater than 4,000) conditions and far enough upstream of the further purification process 10 (i.e., sufficiently prior to the first distillation step 30, such as the point shown by arrow A in FIG. 1) to allow for a residence time of the aromatic diamine and stripped crude MMA stream 20 in the pipe of between 10 and 1200 seconds.

It is well within the ability of persons of ordinary skill in the relevant art, using general engineering principles and empirical studies directed to the particular equipment and apparatus in use, to determine the position upstream of the further purification process 10 that will allow a sufficient residence time necessary to convert enough of the biacetyl present in the stripped crude MMA stream 20 to provide a purified MMA product (i.e, DMMA stream) 35, 45 of less than 2 ppm biacetyl. Of course, how much biacetyl conversion is necessary to achieve an MMA product of less than 2 ppm biacetyl will depend on how much biacetyl is initially present in the stripped crude MMA stream 20. For example, where the stripped crude MMA stream 20 initially comprises 10 ppm biacetyl, by weight, and the desired biacetyl content for the DMMA product (35, 47) is not more than 2 ppm, then it is necessary to provide sufficient residence time in the process equipment and apparatus prior to the first distillation step 30 to react at least 80% ([10−2]/10×100) of the biacetyl. The actual residence may be easily calculated using the volume and flow rates of the process. A second embodiment of the present invention provides a feedback control method for reducing accumulation of solid material in separation and purification equipment in a process for producing a (meth)acrylic acid ester having a biacetyl content of between 0 and less than 2 ppm. Processes which may benefit from application of the feedback control method of the present invention are those where the biacetyl content of the stripped crude (meth)acrylic acid ester stream 20 varies.

For better understanding of the following description, reference may be made back to FIG. 1. The feedback control method of the present invention may suitably be practiced with processes for producing (meth)acrylic acid esters which involve providing a crude, or stripped crude, (meth)acrylic acid ester stream 20 comprising: at least 95% by weight of (meth)acrylic acid ester, not more than 5% by weight of water, and a biacetyl content of not more than 50 ppm, for example, not more than 25 ppm, or even not more than 10 ppm, based on the total weight of the crude (meth)acrylic acid ester stream 20, and adding an aromatic diamine to the crude (meth)acrylic acid ester stream 20 at an addition rate which produces a treated crude (meth)acrylic acid ester stream 20a having an initial molar ratio of aromatic diamine to biacetyl between 1:1 and 100:1, such as not more than 20:1.

The treated crude (meth)acrylic acid ester stream 20a is further purified, in the separation and purification equipment 30 to produce an overhead product 35 which is a purified (meth)acrylic acid ester stream comprising at least 99% by weight (meth)acrylic acid ester, not more than 1% by weight water, and not more than a target value of biacetyl content which is less than the initial biacetyl content, based on the total weight of the purified (meth)acrylic acid ester stream.

For example, the target value of biacetyl content may be between 0 and 5 ppm, by weight, based on the total weight of the purified (meth)acrylic acid ester stream. Furthermore, a purified (meth)acrylic acid ester stream (DMMA) having a biacetyl content of essentially zero, based on non-detection by standard gas chromatography methods, can be achieved without fouling the downstream equipment, in accordance with the present invention. This is accomplished by adjusting the addition rate of aromatic diamine to the point where biacetyl is not detected in the purified (meth)acrylic acid ester stream 35 and the downstream equipment exhibit no signs of fouling (such as, for example, increased reboiler steam chest pressure or decreased cooling efficiency, see Commercial scale Example 4b below). While the measured biacetyl content is non-detectable and the downstream equipment does not exhibit signs of fouling, the addition rate is maintained at its current value. While the measured biacetyl content value is greater than zero (detected), the addition rate is increased. Finally, while the downstream equipment demonstrates signs of fouling, the addition rate is decreased. When the addition rate of the aromatic diamine is adjusted by decreasing the addition rate, the addition rate may be maintained at zero for a period of time and then increased above zero. A residence time between 10 and 1200 seconds for the aromatic amine to contact biacetyl is sufficient. Preferably the aromatic diamine is added at a rate which provides a mole ratio of aromatic amine to biacetyl required to react up to 100% of the biacetyl with the aromatic diamine, based on a residence time of at least 300 seconds. This method minimizes the amount of aromatic diamine fed and consumed to produce DMMA with zero biacetyl and, therefore, also reduces fouling risks associated with the customary practice of providing an excess of aromatic diamine.

More particularly, the feedback method of the present invention involves adjusting the addition rate of the aromatic diamine during the further purification process 10, which is accomplished by monitoring the biacetyl content of the purified (meth)acrylic acid ester stream 35 to obtain a measured value biacetyl content and taking one of the following actions depending upon how the measured value biacetyl content compares to the target biacetyl content. While the measured biacetyl content is between a predetermined lower limit and a predetermined upper limit, the addition rate is maintained at its current value. While the measured biacetyl content value is greater than the upper limit, the addition rate is increased. Finally, while the measured biacetyl content value is less than the lower limit, the addition rate is decreased. When the addition rate of the aromatic diamine is adjusted by decreasing the addition rate, the addition rate may be maintained at zero for a period of time and then increased above zero.

The present invention may, for example, without limitation, involve reacting up to 100%, by weight, of the total biacetyl present in the crude (meth)acrylic acid ester stream 20 with the aromatic diamine prior to performing the further purification 10. As another example, if the crude biacetyl content is not more than 10 ppm, at least 80% by weight of the total weight of biacetyl present in the crude (meth)acrylic acid ester stream, could be reacted with the aromatic diamine to produce a high purity (meth)acrylic acid ester product having less than 2 ppm biacetyl. As another example, if the crude biacetyl content is not more than 3 ppm, at least 40% by weight of the total weight of biacetyl present in the crude (meth)acrylic acid ester stream (20), could be reacted with the aromatic diamine to produce a high purity (meth)acrylic acid ester product (35, 45) having less than 2 ppm biacetyl.

The predetermined lower and upper limits of biacetyl content may be, for example, without limitation, 50% of the target biacetyl content value and 75% of the target biacetyl content value, respectively. For instance, if the biacetyl content of the crude (meth)acrylic acid ester stream 20 is not more than 10 ppm and the target biacetyl content value is not more than 2 ppm, the predetermined lower limit is 1 ppm and the predetermined upper limit is 1.5 ppm. Also, if the target biacetyl content value is 0, then for obvious practical reasons, the predetermined lower limit of biacetyl content would also be 0, and the predetermined upper limit of biacetyl content should be whatever is practically acceptable for the particular product and intended end use, such as 2 ppm, or even 1 ppm.

In some embodiments, optional in-line filtration apparatus (not shown) may be beneficially employed in process streams comprising heavy impurities, such as for example, process streams 37 or 47, to minimize the accumulation rate of solid material in separation and purification equipment. Such filtration apparatus may include, but is not limited to, one or more of cartridge filters, inertial filters, sock filters, strainers, leaf filters, wedge-wire filters, sand filters, filter baskets, and centrifugal separators. If practiced, it is preferred that such filtration apparatus be placed upstream heat exchange equipment such as reboilers, feed-to-bottoms exchangers, and bottoms coolers.

A third embodiment of the present invention provides a method for reversing accumulation of solid material in separation and purification equipment in a process for producing a (meth)acrylic acid ester having a biacetyl content of less than 2 parts per million (ppm). The process for producing a (meth)acrylic acid ester begins with providing a crude (meth)acrylic acid ester stream comprising: at least 95% (meth)acrylic acid ester, not more than 5% water, and not more than 50 ppm initial biacetyl content, by weight, based on the total weight of the crude (meth)acrylic acid ester stream and adding an aromatic diamine to the crude (meth)acrylic acid ester stream at a set addition rate which produces a treated crude (meth)acrylic acid ester stream having an initial molar ratio of aromatic diamine to biacetyl between 1:1 and 100:1. Next, the treated crude (meth)acrylic acid ester stream is distilled in the separation and purification equipment, which produces an overhead product which is a purified (meth)acrylic acid ester stream. The purified (meth)acrylic acid ester stream comprises at least 99% by weight (meth)acrylic acid ester, not more than 1% by weight water, and not more than a target value of biacetyl content which is less than the initial biacetyl content, based on the total weight of the purified (meth)acrylic acid ester stream. The target value of biacetyl content in the purified (meth)acrylic acid ester stream may, for example, be from 0 to 2 ppm biacetyl.

It has been surprisingly discovered that, during operation of such a process for producing a high purity (meth)acrylic acid ester, if fouling (i.e., accumulation of solid material) occurs in the separation and purification equipment, it may be possible to reverse such fouling by significant reduction, or even cessation, of the addition rate of aromatic diamine for a period of time, followed by resuming addition of the aromatic diamine. This method relies on being able to monitor the further purification process 10 and determine whether fouling is occurring or not. As will be obvious to persons of ordinary skill in the art, the surest way to determine whether fouling is occurring is to stop the process, open the equipment and visually inspect the interior surfaces of the equipment for the presence of accumulated solid material on those surfaces. Unfortunately, this is very inefficient and disruptive in a commercial operation, particularly if the solution for removal of the solid material does not require actual manual, physical removal such as by scraping, brushing, chipping, etc., the accumulated solid material from the interior surfaces of the equipment. Thus, monitoring one or more operating conditions of the process that would be indicative of fouling is much more advantageous especially when, as in this third embodiment of the present invention, there is an indirect way of removing the solid material.

For example, without limitation, one possible operating condition that would be indicative of fouling inside equipment such as a heat exchanger or reboiler would be an unintended difference in the temperature of the fluid exiting such equipment. For instance, if a steam heated, shell-and-tube type reboiler is operated to deliver a fluid having an exit temperature of 105° C., the onset of fouling might be first identified by an increase in reboiler steam chest pressure, followed thereafter by a decreasing exit temperature of several degrees Celsius or more. Similarly, if a bottoms cooler is operated to produce a fluid having an exit temperature of 10° C., then if the fluid exiting this cooler were to be monitored and found to be at 13° C., this may indicate the presence of accumulated solid material in the bottoms cooler, which would interfere with the bottoms cooler's capacity to cool the fluid to the desired 10° C. temperature. Moreover, there may be an acceptable operating range for this operating condition, such as a desired exit temperature in a range between 9° C. and 11° C., so that a temperature measured outside this predetermined acceptable range of 9° C. and 11° C., such as 13° C., would indicate a problem with the bottoms cooler (e.g., fouling inside the cooler). As easily determinable by persons of ordinary skill in the relevant art, the operating condition to be monitored should be one that is likely to indicate the presence of accumulated solids therein and will depend upon the particular kind of equipment in use in the process.

Thus, the method of the present invention further requires a step of determining that solid material has accumulated to an unacceptable degree in separation and purification equipment by monitoring at least one operating condition and observing that the operating condition has fallen outside a predetermined acceptable range of values. When such an observation is made, the addition rate of aromatic diamine is reduced and maintained within a range of values less than the set addition rate, for a period of time, until the operating condition is observed to fall within the predetermined acceptable range. In the example discussed above, the predetermined acceptable range for the bottoms cooler was between 9° C. and 11° C. When the temperature of the fluid exiting the bottoms cooler measured 13° C., which falls outside the predetermined acceptable range, it could be concluded that fouling was occurring in the cooler, and the addition rate of the aromatic diamine can be reduced and maintained within a range of values less than the set addition rate, for some period of time. When the exit temperature falls within 9° C. and 11° C. again, the addition rate of aromatic diamine may be raised back up to the set addition rate. It is noted that the range of values less than the set addition rate may include zero, which means that the addition rate of aromatic diamine could be reduced to zero for a period of time.

It has been found, surprisingly, that when fouling occurs in processes for producing (meth)acrylic acid ester in which an excess amount of aromatic amine has been provided to the process to facilitate removal of one or more impurities such as biacetyl, reducing or ceasing the addition of aromatic diamine allows accumulated solid materials to dissolve back into process streams and, thereby, resolve itself. In one embodiment of this method, the purified (meth)acrylic acid ester stream (35) produced is also allowed to accumulate in one or more large rundown tanks over a period of several hours of operation in order to obtain a more uniform biacetyl concentration through blending. If such a blending system is utilized, it is preferred that the rundown tanks be mixed or recirculated to achieve maximum homogeneity. A fourth embodiment of the present invention provides a feed-forward, or proactive, method for reducing accumulation of solid material in separation and purification equipment in a process for producing a (meth)acrylic acid ester having a biacetyl content of less than 2 parts per million (ppm). Processes which may benefit from application of the feed-forward control method of the present invention are those where the biacetyl content of the stripped crude (meth)acrylic acid ester stream 20 varies.

For better understanding of the following description, reference may be made back to FIG. 1. The feed-forward control method of the present invention may suitably be practiced with processes for producing (meth)acrylic acid esters which involve providing a crude, or stripped crude, (meth)acrylic acid ester stream 20 comprising: at least 95% by weight of (meth)acrylic acid ester, not more than 5% by weight of water, and a biacetyl content of not more than 50 ppm (such as, for example, not more than 25 ppm, or even not more than 10 ppm), based on the total weight of the crude (meth)acrylic acid ester stream 20, and adding an aromatic diamine to the crude (meth)acrylic acid ester stream 20 at an addition rate which produces a treated crude (meth)acrylic acid ester stream 20a having an initial molar ratio of aromatic diamine to biacetyl between 1:1 and 100:1, such as not more than 20:1.

The treated crude (meth)acrylic acid ester stream 20a is further purified, in the separation and purification equipment 30 to produce an overhead product 35 which is a purified (meth)acrylic acid ester stream comprising at least 99% by weight (meth)acrylic acid ester, not more than 1% by weight water, and not more than a target value of biacetyl content which is less than the initial biacetyl content, based on the total weight of the purified (meth)acrylic acid ester stream. The target value of biacetyl content in the purified (meth)acrylic acid ester stream may be, for example without limitation, from 0 to 2 ppm biacetyl.

More particularly, the feed-forward method of the present invention involves adjusting the addition rate of the aromatic diamine during the further purification process 10, which is accomplished by monitoring the biacetyl content of the stripped crude (meth)acrylic acid ester stream 20 to obtain a measured value biacetyl content and taking one of the following actions, depending upon how the measured value biacetyl content compares to the target biacetyl content. While the measured biacetyl content is between a predetermined lower limit and a predetermined upper limit, the addition rate of aromatic diamine is maintained at its current value. While the measured biacetyl content value is greater than the upper limit, the addition rate of aromatic diamine is increased. Finally, while the measured biacetyl content value is less than the lower limit, the addition rate is decreased. In addition, the feed-forward control method can target biacetyl content in DMMA of essentially zero based on non-detection by standard gas chromatography methods while preventing solid material accumulation in the downstream equipment. The feed-forward method to achieve zero biactetyl in DMMA and prevent solid material accumulation in downstream equipment requires aromatic diamine addition rates be predefined and specifically matched with biacetyl content of the crude (meth)acrylic acid ester stream 20. The specific ratio of aromatic diamine added to the crude (meth)acrylic acid ester stream 20 comprising biacetyl needed to produce DMMA with zero biacetyl content and prevent solids accumulation in downstream equipment is determined experimentally based on various levels of biacetyl content in the crude (meth)acrylic acid ester stream 20, equipment configuration and operating parameters, such as but not limited to Reynolds number, residence time between aromatic diamine and biacetyl, and temperature.

When the addition rate of the aromatic diamine is adjusted by decreasing the addition rate, the addition rate may be maintained at zero for a period of time and then increased above zero.

Up to 100% by weight, of the total biacetyl present in the crude (meth)acrylic acid ester stream 20 may be reacted with the aromatic diamine, prior to performing the further purification 10.

The predetermined lower and upper limits of biacetyl content may be, for example, without limitation, 50% of the target biacetyl content value and 75% of the target biacetyl content value, respectively. For instance, when the biacetyl content of the crude (meth)acrylic acid ester stream 20 is not more than 10 ppm and the target biacetyl content value is not more than 2 ppm, the predetermined lower limit is 1 ppm and the predetermined upper limit is 1.5 ppm.

It will be understood that the embodiments of the present invention described hereinabove are merely exemplary and that a person skilled in the art may make variations and modifications without departing from the spirit and scope of the invention. All such variations and modifications are intended to be included within the scope of the present invention.

Specific applications of the method of the present invention will now be described in the context of the following laboratory and commercial-scale examples.

### EXAMPLES

### Laboratory Example 1

A volume of stripped crude MMA (nominal 95-96% purity and comprising about 5000 ppm MAA) (“SCMMA”) was drawn from a commercial scale ACH-Based manufacturing process and found to have a biacetyl content of about 2.4 ppm, as measured by gas chromatograph (“GC”) analysis. This material was used to produce the following three mixtures:

(a) 50 ml of SCMMA was charged to a capped, 100 ml flask equipped with a stir bar; to this was added a 0.98% stock solution of ortho-phenylenediamine (“oPD”) in SCMMA, so that the molar ratio of oPD to Biacetyl was 10:1. The mixture was allowed to stir at ambient temperature over a period of about 7 hours with periodic sampling and determination of Biacetyl concentration by GC.
(b) Similarly, 50 ml of SCMMA was charged to a second 100 ml flask equipped with a chilled water (7.7 C) condenser, a drying tube, and a stir bar; to this sample was added a stock solution of oPD in SCMMA in sufficient quantity to achieve a molar ratio of oPD to biacetyl of about 10:1. This second mixture was allowed to stir at 50 C over a period of about 6 hours with periodic sampling and determination of Biacetyl concentration by GC.
(c) A third 50 ml mixture was prepared in the same manner as in (b) above. This third mixture was allowed to stir at 80 C over a period of about 5 hours with periodic sampling and determination of Biacetyl concentration by GC.

The first sample of the series from each of these three mixtures was drawn and analyzed as rapidly as possible (less than 5 minutes residence time); GC analysis showed biacetyl content to be below the detection limit (essentially zero) on all three samples. All subsequent samples were also found to be below detection limits. This indicates that biacetyl is rapidly converted to a heavy component (i.e., having a boiling point higher than MMA) and that this biacetyl conversion reaction is not reversible over 5 hours at ambient temperature, over 6 hours at 50 C, nor over 7 hours at 80° C. Additionally, no precipitates or solids accumulations were observed in the test samples.

### Laboratory Example 2

The three mixtures described in Laboratory Example 1 were reproduced, with the exception that the quantity of stock oPD solution used was of sufficient quantity to achieve a molar ratio of oPD to biacetyl of about 5:1. As before, the initial samples (less than 5 minutes residence time) were found to be below detection limits, the biacetyl conversion reaction was found to be not reversible after 5 or more hours, and no precipitates or solids accumulations were observed in the test samples.

### Laboratory Example 3

The three mixtures described in Example 1 were again reproduced, with the exception that the quantity of stock oPD solution used was of sufficient quantity to achieve a molar ratio of oPD to biacetyl of about 2:1. As in the previous examples, the initial samples (less than 5 minutes residence time) were found to be below detection limits, the biacetyl conversion reaction was found to be not reversible after 5 or more hours, and no precipitates or solids accumulations were observed in the test samples.

### Laboratory Example 4

In the production of DMMA via distillation, a bottoms stream is also produced comprising heavy impurities and MMA (see FIG. 1, heavies stream 37). This bottoms stream may be further processed in a stripping column (40, FIG. 1) to recover residual MMA. Such processing may subject the bottoms stream to temperatures of up to 125° C. for extended periods of time. To assess the effects of such elevated temperatures, and the presence of concentrated heavy impurities, on the stability of the heavy compounds formed by the biacetyl conversion reaction, a sample of the MMA-depleted bottoms material from such a stripping operation (said bottoms material herein referred to as “TSB”) was collected for experimentation. In a similar fashion to the previous experiments, a volume of TSB was spiked to achieve a 115 ppm concentration of biacetyl and then subsequently treated with a sufficient quantity of stock oPD solution to achieve a molar ratio of oPD to biacetyl of about 2:1. This treated material was continuously mixed, heated to 125° C. and maintained at that temperature for 8 hours. As in the previous examples, the initial samples (less than 5 minutes residence time) were found to be below detection limits, the biacetyl conversion reaction was found to be not reversible over the 8 hour time frame, and no precipitates or solids accumulations were observed in the test sample.

As discussed earlier herein, despite the excellent biacetyl removal performance of oPD shown in the foregoing Laboratory Examples 1-4, application of this purification technology to a commercial-scale process for producing MMA surprisingly fell short of expectations, with instances of incomplete biacetyl removal and fouling of heat transfer surfaces within the manufacturing process.

### Commercial-Scale Examples

In each of the following trials, a commercial-scale MMA distillation system was utilized to treat and distill actual production-quality stripped crude MMA. The objective of these trials was to demonstrate the conditions under which commercial-grade distilled MMA product (DMMA) comprising not more than 2 ppm biacetyl could be produced over long periods of time without significant fouling of the separation and purification (e.g., distillation equipment and ancillaries such as reboilers, condensers, etc.).

FIG. 2 provides a schematic diagram of the commercial-scale MMA distillation system 300 with which the following experimental trials were performed. The commercial-scale MMA distillation system 300 was used to perform the first distillation step 30 of a commercial-scale MMA production process similar to that described above in connection with FIG. 1. The distillation system 300 was used to remove high boiling impurities (also known as “heavy-ends”) from an SCMMA stream (20, FIG. 1) produced by a conventional ACH-based MMA process and an associated stripping step. As used herein, the term SCMMA means a partially-purified crude MMA stream, comprising about 95-96% MMA, from which a quantity of low-boiling impurities, such as for example water and methanol have already been removed in a removal step (20, FIG. 1).

The distillation system 300 included a vacuum distillation column 310, an overhead condenser supplied with cooling tower water 302, a hydroquinone (“HQ”) inhibitor solution feed tank 303, a steam heated, continuous-circulation external reboiler 304, a feed-to-bottoms heat exchanger 305, and a bottoms cooler supplied with refrigerated cooling water 307. Ancillary equipment such as pumps, filters, control valves, and the like were also present, but have been omitted from FIG. 2 for simplicity and clarity.

The distillation column 310 had 20 internal sieve trays with downcomers.

Hereinafter, the term “Tray 1” means the bottom-most tray of the column 310, and “Tray 20” means the top-most tray in the column 310. A vacuum system connected to the column (not shown) maintains column top pressure at about 240 mmHg. The flow rate of ambient temperature SCMMA to be purified was controlled by adjustment of the feed flow control valve 301. After passing through the feed flow control valve 301, the SCMMA was preheated in the feed-to-bottoms exchanger 305 to a temperature of between 30° C. and 36° C. and then entered the distillation column 310 via a feed nozzle (not shown per se) aligned with feed Tray 6 (306) in the column 310. A solution of hydroquinone (HQ) inhibitor dissolved in MMA was drawn from the inhibitor feed tank 303 and fed onto Tray 18 (318). Air (not shown) was also added to the bottom of the column 310 to maintain efficacy of the HQ inhibitor. Distilled MMA vapor is drawn from the top of the column 310 and condensed in the overhead condenser 302. A portion of the condensate 309 thus formed is returned to the column 310 (reflux) and a portion 311 is sent to storage (rundown) as DMMA Product.

The reboiler (304) maintained the temperature at the bottom of the column between 80° C. and 90° C. Bottoms material 370 comprising heavy-ends impurities was drawn from the bottom of the column 310, passed through the feed-to-bottoms exchanger 305 for initial cooling to about 35° C., and then further cooled in the bottoms cooler 307, where the bottoms stream temperature was reduced to about 8° C. to 10° C. in order to minimize organic vapor emissions in downstream storage tanks (not shown). In some of the Examples, solution containing oPD is stored in a temporary feed tank 308, shown in phantom in FIG. 2.

% Biacetyl conversion to heavy compound(s) is calculated as follows:

$$100 \times \frac{[ \begin{matrix}( initial ppm Biacetyl in SCMMA ) - \\ ( final ppm Biacetyl in DMMA )\end{matrix} ]}{( initial ppm Biacetyl in SCMMA )}$$

oPD:Biacetyl molar treatment ratio is defined as follows:

$$\frac{( \# moles oPD added )}{( initial moles biacetyl in SCMMA to be treated )}$$

### Commercial-Scale Example 1

A 4.5 wt % oPD in DMMA solution was prepared and placed into the temporary feed tank 308. The tank 308 was connected by temporary tubing to a point immediately upstream of the distillation column feed flow control valve 301, which is itself a short distance upstream of the feed-to-bottoms heat exchanger 305. The oPD solution was added directly to the SCMMA feed line at a constant rate of 6 gph.

As configured, the region within which the oPD and MMA could be mixed and have residence time comprised the approximately 45 linear feet of 4-inch, schedule 40 piping and an 85 sq. ft. spiral feed-to-bottoms heat exchanger 305 located between the feed flow control valve and the distillation column Tray 6 feed nozzle.

At the SCMAA feed rate of 68,000 pounds/hour used throughout this trial, fully turbulent flow was developed within the piping (Reynolds number>200,000), providing thorough mixing of the oPD and SCMMA. Additionally, the spiral feed-to-bottoms heat exchanger also provided thorough mixing as it is designed to maximize turbulence for enhanced heat transfer. Thus, a liquid phase residence time for biacetyl in SCMMA of about 24 seconds was provided before the mixed treated SCMMA stream entered the distillation column.

The SCMMA had biacetyl concentration of 2.5 ppm and comprised between 0.3 and 0.5% MAA. The resulting oPD:Biacetyl molar ratio was 10.6:1. Samples of the DMMA product 311 showed no detectable biacetyl present (biacetyl content=0 ppm).

The trial progressed for 84 hours until the oPD solution in the temporary feed tank was depleted. At the end of the trial, it was noted that the bottoms cooler 307 had become rapidly fouled, since the bottoms outlet temperature increased from its normal range of about 8° C.-10° C. up to 12° C.-13° C. during this relatively brief 84 hour test period.

### Commercial-Scale Example 2a

This next trial was also performed using the previously-described distillation system shown in FIG. 2 and described above. During this 16.5-hour trial period, the column was continuously fed 68,000 pounds/hour of SCMMA with an average biacetyl concentration of 3.4 ppm.

In this trial, approximately 25 pounds of oPD were added to the distillation system HQ inhibitor solution feed tank 303 (nominal 1.5 wt % HQ in DMMA) and mixed to produce a volume of HQ inhibitor solution comprising 1.31% oPD. Over the course of the trial, two ‘make-up’ additions of fresh HQ and DMMA were made to gradually lower the concentration of oPD in the inhibitor tank.

The oPD-containing inhibitor solution was pumped at a continuous flow rate of about 19 gallons/hour through a feed nozzle located immediately above Tray 18 of the distillation column. At the start of the trial, the delivery of 1.31% oPD solution to the distillation column in this manner equated to an oPD concentration of about 30.5 ppm within the distillation column, for an initial oPD:biacetyl molar ratio of 7.1:1. The results of Commercial-scale Examples 2a (cases I, ii and iii), 2b and 2c are shown below in Table 1.

TABLE 1

| | oPD conc | oPD conc | oPD:Bi- | Biacetyl conc | Biace- |
|---|---|---|---|---|---|
| Mix- | in inhibitor | in distilla- | acetyl mo- | in DMMA | tyl con- |
| ture | solution | tion column | lar ratio | product | version |
| Std. | 0 | 0 | N/A | 3.4 ppm | N/A |
| (a) | 1.31 wt % | 30.5 ppm | 7.1:1 | 2.5 ppm | 26% |
| (b) | 0.87 wt % | 20.3 ppm | 4.7:1 | 2.6 ppm | 24% |
| (c) | 0.64 wt % | 14.9 ppm | 3.5:1 | 2.8 ppm | 18% |

During this relatively brief trial run, signs of rapid fouling were seen in the bottoms cooler 307, since the bottoms outlet temperature increased from its normal range of about 8° C.-10° C. up to 11° C.-12° C..

This trial demonstrated that adding oPD onto the top surface of a distillation tray at molar ratios from 3.5:1 up to 7:1 is not effective at reducing biacetyl content from 3.4 ppm to 2 ppm or less and also leads to fouling of distillation system heat transfer equipment.

Given the rapid and highly efficient removal of biacetyl in the laboratory upon addition of oPD, this poor performance at the commercial scale was very surprising. Without wishing to be bound by theory, it is hypothesized that the low Biacetyl:heavy compound conversion achieved during this trial may be related to insufficient residence time of liquid phase biacetyl on the distillation tray (estimated to average less than 10 seconds) and possibly also due to insufficient mixing.

### Commercial-Scale Example 2b

As follow-up to the previous trial, the oPD solution used in Commercial-Scale Example 2a was tested in the laboratory to verify its effectiveness. An SCMMA sample containing 2.5 ppm biacetyl was treated at ambient temperature with sufficient oPD solution to obtain a 2:1 oPD:biacetyl molar treatment ratio and shaken well to thoroughly mix. Within 5 minutes, a sample of this treated mixture was analyzed by GC and resulted in measurements below detection limits (<1 ppm) for biacetyl concentration. This demonstrated that the oPD solution used in Commercial-Scale Example 2a was active and capable of rapidly converting biacetyl to heavy compound(s) to effectively facilitate removal of biacetyl from the MMA.

### Commercial-Scale Example 2c

Another trial was performed in which the previously-described distillation system of FIG. 2 was continuously fed 68,000 pounds/hour of SCMMA. In this trial, the SCMMA had an average biacetyl concentration of 2.5 ppm.

Sufficient oPD was mixed into the HQ inhibitor solution tank to produce a volume of HQ inhibitor solution comprising 1.00% oPD, 1.5% HQ, and the balance MMA. During this trial, which spanned about 110 hours, the concentration of oPD in the inhibitor solution remained constant.

The oPD-containing inhibitor solution was pumped at a continuous flow rate of about 22 gallons/hour through a feed nozzle located immediately above Tray 18 of the distillation column. At these conditions, the column operated at an oPD:biacetyl molar ratio of 8.6:1.

In this trial, however, biacetyl to heavy compound conversion was only 8% and the biacetyl concentration in the DMMA product was outside of specifications at an average of 2.3 ppm. Additionally, signs of rapid fouling were again seen in the bottoms cooler, since the bottoms outlet temperature was observed to increase from its normal range of about 8° C.-10° C. up to 12° C.-14° C. Fouling was also clearly observed in the reboiler apparatus.

Given the poor biacetyl removal efficiency in this trial, notwithstanding the use of increased oPD:biacetyl molar ratios, insufficient mixing and residence time were again suspected as key factors. Without wishing to be bound by theory, it was suspected that mass-transfer limitations play a more significant role as initial biacetyl concentrations decrease, making it all the more important to provide thorough mixing between oPD and biacetyl in the MMA stream. It was also hypothesized that the low Biacetyl:heavy compound conversion experienced during this trial may be related to insufficient residence time of liquid phase biacetyl on the distillation tray (estimated to average less than 10 seconds) and possibly insufficient mixing.

### Commercial-Scale Example 3

The previously-described commercial-scale distillation system was utilized for a third trial, lasting 11 days. During this trial period, the distillation column was continuously fed a stream of SCMMA with an average biacetyl concentration of 2.5 ppm at a feed rate of 68,000 pounds/hour. An oPD solution comprising 1% oPD and 1.5% HQ dissolved in DMMA was mixed in the inhibitor feed tank and then fed to the distillation system simultaneously at two locations at a combined feed rate of 41 gallons/hour. More particularly, the solution was added at a rate of 22 gallons/hour directly to Tray 18 of the distillation column (i.e., in the same manner as Commercial-scale Examples 2a and 2c), and the solution was also added at a rate of 19 gallons/hour to the SCMMA Feed line at a point immediately upstream of the feed flow control valve (i.e., in the same manner as Commercial-scale Example 1). Under these conditions, the distillation system operated with an oPD:biacetyl molar treatment ratio of 16:1. Samples of the DMMA product were regularly analyzed over the course of the trial period and were determined to have an average biacetyl concentration of 1.5 ppm, which equates to a 40% biacetyl:heavy compound conversion. Over the trial period, the feed-to-bottoms exchanger 305 showed signs of rapid fouling, with the bottoms outlet temperature increasing from its normal temperature of about 35° C. to more than 50° C. (above the normal span of this temperature indicator). Similar signs of fouling were seen in the bottoms cooler 307 as well, i.e., the bottoms outlet temperature increased from its normal range of about 8° C.-10° C. up to 18° C.-22° C. Fouling was also clearly observed in the reboiler apparatus, at which point this trial run was discontinued.

This trial demonstrated that, although the biacetyl specification was met for DMMA, operation at an oPD:biacetyl molar ratio greater than 10:1 led to rapid fouling of the distillation system heat transfer surfaces.

### Commercial-Scale Example 4

A fourth and final series of trials were undertaken to verify the operating parameters identified in earlier work under long-term commercial-scale operating conditions. These trials were performed with varying oPD:Biacetyl ratios to better define the operating range and to demonstrate the reversibility of heat transfer surface fouling over a range of operating conditions.

For this trial, SCMMA was sourced from a large-volume (greater that 1 million pounds capacity) intermediate storage tank to ‘buffer’ potential variations in SCMMA biacetyl concentration. Over the trial period, the SCMMA stream averaged 95-96% by weight MMA, between 0.3% and 0.5% by weight MAA, and less than 5 ppm biacetyl. As in the previous examples, the ultimate objective of the trial was to demonstrate the ability to produce a DMMA product that meets the biacetyl content specification of less than 2 ppm while simultaneously minimizing fouling of the heat transfer equipment within the distillation system.

In these trials, a feedback-control operating philosophy was applied in which an initial oPD:biacetyl molar treatment ratio and a target DMMA biacetyl concentration was first selected and then the flow of oPD solution was adjusted, based upon monitoring of the actual biacetyl content of the DMMA, to maintain the biacetyl concentration at the target value.

This approach allows the actual oPD:Biacetyl mole ratio in the column to be corrected to accommodate changes in the biacetyl concentration of the SCMMA being fed to the distillation system, which is known to occur over time during normal continuous operations. Such changes in the biacetyl concentration may occur for many reasons, including differences in SCMMA process manufacturing rate and operating conditions, or sourcing from multiple manufacturing facilities, and may be so gradual as to be only detectable over long periods of operation. For this reason, these final trials were extensive and covered a period of 6 months.

During these trials, the DMMA biacetyl content was monitored by regular sampling of the DMMA product rundown 311 and GC analysis. This monitoring could also have been accomplished using (continuous) process analyzers, e.g., online GC or FTIR devices.

In order to limit fouling from overfeeding oPD, it was decided to target 80% conversion of biacetyl to heavy compound(s) and to employ a lower control value (LCV) of 90% conversion. For an SCMMA stream with a biacetyl content of 5 ppm, this equates to a DMMA biacetyl target value of 1 ppm and an LCV of 0.5 ppm. For convenience, the upper control value (UCV) was set at 1.5 ppm. It should be noted, however, that it is not strictly necessary for the range of control values (UCV, LCV) to be numerically symmetric about the biacetyl target value.

Although not implemented for this series of experiments, the use of automation to maintain oPD flow in ratio to the SCMMA feed flow would also be advantageous for long term commercial operation. It is envisioned that a feed-forward scheme (wherein the biacetyl content in the SCMMA feed is monitored and used to make oPD usage adjustments) could also be beneficially employed.

A constant-composition of oPD solution was used throughout the trial, comprising 3.4 wt % oPD, 200 ppm phenothiazine (“PTZ”), and DMMA as solvent. This oPD solution was contained in a temporary feed tank 308 and was added directly to the SCMMA feed line at a point immediately upstream of the feed flow control valve (in the same manner as Commercial-scale Example 1). The region within which the oPD and MMA could be mixed and have residence time comprised the approximately 45 linear feet of 4-inch, schedule 40 piping and an 85 sq. ft. spiral feed-to-bottoms heat exchanger 305 located between the feed flow control valve 301 and the distillation column Tray 6 feed nozzle. Fully turbulent flow within the piping and the spiral exchanger provided thorough mixing of the oPD into the SCMMA. At the SCMAA feed rate of 68,000-70,000 pounds/hour used throughout this trial, this region provided a liquid phase residence time about 24 seconds before the treated stream entered the distillation column. The results are summarized in Table 2 below.

TABLE 2

| | oPD:Biacetyl | Avg ppm | Test | Bottoms Cooler Ref. Water Supply vs. Return: | | | |
|---|---|---|---|---|---|---|---|
| Trial | Molar Treat- | Biacetyl | Period | Ave Temperature Difference (in C.) | | | Observations re: Heat Transfer Surface |
| # | ment Ratio | in DMMA | (hours) | Prior to test | During test | After test | Fouling |
| 4a | 5.7:1 | 0.86 | 166 | 24.11 +/− 1.45 | 24.33 +/− 1.69 | — | No statistical difference in |
| | | | | 35 hrs prior | 166 hr ave | | Temperatures = No Fouling |
| 4b | 7.4:1 | None | 18 | 24.26 +/− 1.83 | 24.36 +/− 1.82 | — | No statistical difference in |
| | | Detected | | 18 hrs prior | 18 hr ave | | Temperatures = No Fouling |
| 4c1 | 12.4:1 | None | First 24 | 23.75 +/− 2.40 | 21.46 +/− 2.71 | — | Statistically Significant difference |
| | | Detected | of 80 | 24 hrs prior | First 24 hrs only | | in Temperatures = Fouling of Heat |
| | | | | | | | Transfer Surface |
| 4c2 | 12.4:1* | None | Last 24 | — | 22.34 +/− 2.18 | 24.26 +/− 2.54 | Statistically Significant difference |
| | | Detected | of 80 | | Last 24 hrs only | 24 hrs after | in Temperatures = Fouling is Revers- |
| | | | | | | | ible when oPD removed |

*The oPD feed rate for this Example 4c2 was periodically maintained at zero and, therefore, this value of molar ratio of oPD:biacetyl represents only the molar ratio achieved while the oPD flow rate was greater than zero. The actual molar ratio achieved during this example was, of course, a value less than 12.4:1, but greater than 0:1.

During the actual testing period, the SCMMA biacetyl content was regularly analyzed and found to average about 2.61 ppm. The average DMMA biacetyl content was about 0.98 ppm during the test period, which equates to an average biacetyl conversion to heavy compound(s) of about 62%.

What is claimed is:

1. A method for reducing accumulation of solid material in separation and purification equipment in a process for producing a (meth)acrylic acid ester having a biacetyl content of less than 2 parts per million (ppm), the process comprising:
A) providing a crude (meth)acrylic acid ester stream comprising: at least 95% (meth)acrylic acid ester, not more than 5% water, and not more than 50 ppm biacetyl, by weight, based on the total weight of the crude (meth)acrylic acid ester stream;
B) adding an aromatic diamine to the crude (meth)acrylic acid ester stream at an addition rate which produces a treated crude (meth)acrylic acid ester stream having an initial molar ratio of not more than 10:1 of aromatic diamine to biacetyl;
C) reacting at least a portion of the total biacetyl present in the crude (meth)acrylic acid ester stream with the aromatic diamine; and
D) subsequent to step C), distilling the treated crude (meth)acrylic acid ester stream, in the separation and purification equipment, to produce an overhead product which is a purified (meth)acrylic acid ester stream comprising at least 99% by weight (meth)acrylic acid ester, not more than 1% by weight water, and less than 2 ppm biacetyl, based on the total weight of the purified (meth)acrylic acid ester stream;
wherein said step C) is performed prior to distilling the treated crude (meth)acrylic acid stream by.
C1) adding the aromatic amine far enough upstream of the separation and purification equipment to provide a residence time of between 10 and 1200 seconds for the aromatic amine to contact biacetyl in the crude (meth)acrylic acid ester stream before performing step D) distilling; and
C2) thoroughly mixing the aromatic diamine with the crude (meth)acrylic acid ester stream.

2. The method according to claim 1, wherein the residence time provided is between 10 and 600 seconds.

3. The method according to claim 1, wherein said step C2) of thoroughly mixing the aromatic amine and crude (meth)acrylic acid ester stream is accomplished by at least one of the following techniques:
a) operating the process with a flow rate of crude (meth)acrylic acid ester stream sufficient to provide turbulent flow conditions, which comprises having a Reynolds number greater than 4000, in the process equipment, and
b) providing the crude (meth)acrylic acid stream and the aromatic amine, or the treated crude (meth)acrylic acid ester stream, to apparatus positioned upstream of the separation and purification equipment and having mixing means comprising one or more static mixers, baffles, recirculation loops, agitators, powered in-line mixers, and mechanical mixers.

4. The method according to claim 3, wherein said apparatus positioned upstream of the separation and purification equipment comprises a vessel, a pipe, a conduit, a tank, or a combination thereof.

5. The method according to claim 1, wherein the aromatic diamine comprises at least one compound selected from the group consisting of: ortho-phenylenediamine, para-phenylenediamine, and meta-phenylenediamine.

6. The method according to claim 1, wherein the aromatic diamine comprises ortho-phenylenediamine.

7. The method according to claim 1, wherein said (meth)acrylic acid ester is methyl methacrylate.

8. The method according to claim 1, wherein the molar ratio of aromatic diamine to biacetyl is not more than 2:1.

9. The method according to claim 1, wherein step B) of adding the aromatic diamine is accomplished by adding to the crude (meth)acrylic acid ester stream a solution comprising a solvent and from 0.5% to 8% by weight of ortho-phenylenediamine, based on the total weight of the solution, wherein said solvent is the same as said (meth)acrylic acid ester.

10. A method for reducing accumulation of solid material in separation and purification equipment in a process for producing a (meth)acrylic acid ester having a biacetyl content of less than 2 parts per million (ppm), the process comprising:
A) providing a crude (meth)acrylic acid ester stream comprising: at least 95% (meth)acrylic acid ester, not more than 5% water, and not more than 50 ppm initial biacetyl content, by weight, based on the total weight of the crude (meth)acrylic acid ester stream;
B) adding an aromatic diamine to the crude (meth)acrylic acid ester stream at an addition rate which produces a treated crude (meth)acrylic acid ester stream having an initial molar ratio of aromatic diamine to biacetyl between 1:1 and 100:1;
C) distilling the treated crude (meth)acrylic acid ester stream, in the separation and purification equipment, to produce an overhead product which is a purified (meth)acrylic acid ester stream comprising at least 99% by weight (meth)acrylic acid ester, not more than 1% by weight water, and not more than a target value of biacetyl content which is less than the initial biacetyl content, based on the total weight of the purified (meth)acrylic acid ester stream; and
D) adjusting the addition rate of the aromatic diamine during operation of the separation and purification equipment by:
(i) monitoring the biacetyl content of the purified (meth)acrylic acid ester stream to obtain a measured value biacetyl content; and
(ii) taking one of the following actions depending upon how the measured value biacetyl content compares to the target biacetyl content;
(a) maintaining the addition rate at its current value while the measured biacetyl concentration is between a predetermined lower limit and a predetermined upper limit;
(b) increasing the addition rate when the measured value biacetyl content is greater than the upper limit; and
(c) decreasing the addition rate when the measured value biacetyl content is less than the lower limit.

11. The method according to claim 10, wherein when the addition rate of the aromatic diamine is adjusted by decreasing the addition rate, the addition rate is maintained at zero for a period of time and then increased above zero.

12. The method according to claim 10, wherein the (meth)acrylic acid ester is methyl methacrylate.

13. The method according to claim 10, wherein the aromatic diamine comprises at least one compound selected from the group consisting of: ortho-phenylenediamine, para-phenylenediamine, and meta-phenylenediamine.

14. The method according to claim 10, wherein the predetermined lower limit is 50% of the target value biacetyl content and the predetermined upper limit is 75% of the target value biacetyl content.

15. A method for reversing accumulation of solid material in separation and purification equipment in a process for producing a (meth)acrylic acid ester having a biacetyl content of less than 2 parts per million (ppm), the process comprising:
A) providing a crude (meth)acrylic acid ester stream comprising: at least 95% (meth)acrylic acid ester, not more than 5% water, and not more than 20 ppm initial biacetyl content, by weight, based on the total weight of the crude (meth)acrylic acid ester stream;
B) adding an aromatic diamine to the crude (meth)acrylic acid ester stream at a set addition rate which produces a treated crude (meth)acrylic acid ester stream having an initial molar ratio of aromatic diamine to biacetyl between 1:1 and 100:1;
C) distilling the treated crude (meth)acrylic acid ester stream, in the separation and purification equipment, to produce an overhead product which is a purified (meth)acrylic acid ester stream comprising at least 99% by weight (meth)acrylic acid ester, not more than 1% by weight water, and not more than a target value of biacetyl content which is less than the initial biacetyl content, based on the total weight of the purified (meth)acrylic acid ester stream; and
D) determining that solid material has accumulated to an unacceptable degree in the separation and purification equipment by monitoring at least one operating condition and observing said at least one operating condition falling outside a predetermined acceptable range; and
E) reducing and maintaining the addition rate of aromatic diamine within a range of values less than the set addition rate, for a period of time until said at least one operating condition is observed to fall within said predetermined acceptable range.

16. The method according to claim 15, wherein said range of values less than the set addition rate has a lower limit of zero.

17. The method according to claim 15, wherein the overhead product which is a purified (meth)acrylic acid ester stream is accumulated and blended in one or more tanks to homogenize the biacetyl concentration therein.
